(19) United States
(12) Patent Application Publication
(10) Pub. No.: US 2015/0100050 A1
(43) Pub. Date: Apr. 9, 2015

(54) LASER CAPSULOTOMY DEVICE AND METHOD OF USE

(71) Applicant: LUMENIS LTD., Yokneam Ilit, IL (US)

(72) Inventors: Kfir Azoulay, Amsterdam (NL); Asaf Bar, Santa Clara, CA (US)

(21) Appl. No.: 14/507,949

(22) Filed: Oct. 7, 2014

Related U.S. Application Data
(60) Provisional application No. 61/888,228, filed on Oct. 8, 2013.

(51) Int. Cl. A61F 9/008

(52) U.S. Cl. CPC A61F 9/00836; A61F 2009/00878; A61F 2009/0087; A61F 2009/00897; A61F 2009/00889; A61F 2009/00844
USPC 606/6

(57) ABSTRACT

An apparatus and method for treating opacity in the posterior eye capsule includes using an illumination device to identify and analyze tension lines in the posterior eye capsule and then using a laser to perforate at least a portion of the posterior eye capsule along the tension lines.

### RELATED APPLICATIONS

This application claims priority to U.S. Provisional Application Ser. No. 61/888,228, filed Oct. 8, 2013. The full disclosure of each of these provisional applications is incorporated by reference herein.

### FIELD OF THE INVENTION

This invention relates to opthamology and in particular to the treatment of the eye following cataract surgery due to opacification of the posterior capsule.

### BACKGROUND

After a cataract operation, opacification of the posterior capsule may occur at the opposite of the anterior capsule flap. Posterior capsule opacification is a result of the formation of opaque secondary membranes by active lens epithelial proliferation, transformation of lens epithelial cells into fibroblasts with contractile elements, and collagen synthesis and deposition. Collagen deposition results in white fibrotic tissue which reduces posterior capsular clarity dramatically. Moreover, contraction caused by the deposited collagen produces wrinkling and the presence of tension lines in the posterior capsule.

Fibrosis occurring during the first days or weeks postoperatively most often represents cortical lamellae left at the time of surgery and may result in broad undulations of the clear capsule, before it becomes tense, and is rarely visually disturbing to the patient. However, fibrosis that develops months to years postoperatively is caused by migration of anterior lens epithelium and collagen production which establishes a dense fibrinous plaque which increases opacity. A second type of opacity may occur months to years postoperatively due to proliferating lens epithelial cells which may form layers several cells thick which acquire contractile properties and causing visually disturbing fine wrinkles

Laser capsulotomy is popularly indicated for the treatment of opacification of the posterior capsule. FIG. 1 illustrates a “before and after” view of the eye prior to and after laser capsulatomy. The laser, usually an Nd:YAG, is delivered into the eye and to the posterior capsule through a contact lens which increases the laser beam convergence angle. In this way the spot size is big enough and hence the energy fluence is low enough on both cornea and the retina, while the minimal amount of energy necessary to obtain breakdown and rupture of the posterior capsule is maintained. A typical capsule can be opened by using one or more 1-2 mJ/pulses.

In order to successfully perform a capsulotomy with the lowest number of laserpulses, the physician has to visually examine the posterior capsule for fine wrinkles that indicate tension lines. Then laser shots are placed across the tension lines in a cruciate pattern. Preferably, the physician starts at 12 o'clock and continues downward to 6 o'clock and then shoots the laser laterally from 3 o'clock to 9 o'clock Aiming the laser pulses across tension lines results in the largest opening per pulse because the tension causes the initial opening to widen.

U.S. Pat. No. 4,495,665 teaches an intraocular lens having plurality of spaced projections at its interface with the posterior capsule configured to press the posterior capsule in order to cause concentrate line of tension between such projections, so that increased tear of the posterior capsule will occur after laser perforation of the capsule in said region.

Barman et al, in “Quantification of Posterior Capsular Opacification in Digital Images after Cataract Surgery”, Invest Ophthalmol Vis Sci. 2000: 41:3882-3892 teach a retroillumination camera that produces high-resolution digital images of the posterior capsule that can demonstrate the proliferation of lens epithelial cells in an opacified eye. Local variances of pixel density indicate texture changes providing objective measure of posterior capsular opacification.

### SUMMARY OF THE PRESENT INVENTION

According to a first aspect of the invention, there is provided a laser capsulotomy device comprising a camera operable to acquire an image of at least a portion of a patient's posterior capsule, a display to display the acquired image, displaying at least a portion of tension lines in the image, and a treatment laser, the treatment laser being operable to direct at least one laser pulse to perforate the posterior capsule.

According to another aspect of the present invention there is provided a method of treating a secondary cataract in a patient eye using an embodiment of the laser capsulotomy device based on the present invention. The method includes acquiring an image of at least a portion of a patient's posterior capsule, displaying at least a portion of the tension lines in such image, targeting a treatment laser to posterior capsule of such patient and shooting at least one laser pulse aiming to perforate the posterior capsule.

According to another aspect of the present invention the method further includes perforating the posterior capsule of a patient's eye along at least one line which is approximately perpendicular to the main axis of the tension lines.

According to yet another aspect of the present invention, the method of treatment further comprising illuminating the at least a portion of the posterior capsule of a patient's eye. Illuminating the posterior capsule may be done by changing optical characteristics of the illuminating light such as intensity, duration, frequency, angle or pulsation,

According to another aspect of the present invention, the method further includes acquiring images of the posterior capsule at different illumination profiles and identify optical changes based on the analysis of at least one of the absorption, reflection, scattering or diffraction of the illuminated or back scattered light and identifying the location or orientation of tension line on the posterior capsule.

According to yet another aspect of the present invention an image processing software may further enhance the identification or location and orientation of such tension lines.

The details of one or more variations of the subject matter described herein are set forth in the accompanying drawings and the detailed description below. Other features and advantages of the subject matter described herein will be apparent from the description and drawings as well the appended claims herein.

In another aspect, a method of performing a laser capsulotomy on a posterior capsule includes providing a laser; illuminating at least a portion of the posterior capsule; acquiring at least one image of the illuminated posterior capsule; analyzing the image of the acquired image; identifying optical changes based on one or more of: absorption, reflection, scattering of illuminated or back-scattered light; identifying one or more of the location and orientation of one or more tension lines formed on the posterior capsule; displaying one or more images of the tension lines identified; and, perforating at least a portion of the posterior capsule along at one or more of the tension lines.

In another aspect, a plurality of images at different illumination profiles are acquired during the step of acquiring an image of the illuminated posterior capsule.

In another aspect, acquiring an image of the illuminated posterior capsule may include changing the optical characteristics of the illuminating light.

In yet another aspect, the changing of optical characteristics may include changing one or more of: intensity, duration, frequency, angle or pulsation.

In yet another aspect, there may be processing of the one or more optical characteristics to produce multiple images of the posterior capsule based on the different illumination characteristics.

In another aspect, perforating the posterior capsule comprises perforating along one or more lines which are approximately perpendicular to a main axis of the one or more tension lines.

In another aspect, there is disclosed an apparatus for performing a laser capsulotomy on a posterior eye capsule having: a laser source; a controller to control the laser source; the controller has a processor and a non-transitory computer-readable medium; the non-transitory computer-readable medium stores instructions that, when executed by the processor, cause the processor to: illuminate at least a portion of the posterior capsule; acquire at least one image of the illuminated posterior capsule; analyze the image of the acquired image; identify optical changes based on one or more of: absorption, reflection, scattering of the illuminated or back-scattered light; identify one or more of the location and orientation of one or more tension lines formed on the posterior capsule; display one or more images of the tension lines identified on a display; and then to perforate at least a portion of the posterior capsule along at one or more of the tension lines.

In another aspect, the laser source is a Nd:YAG laser.

In another aspect, the posterior capsule is perforated along one or more lines which are approximately perpendicular to a main axis of the one or more tension lines.

In yet another aspect, a method for performing a laser capsulotomy on a posterior eye capsule includes: illuminating the posterior eye capsule to identify one or more tension lines formed in the posterior eye capsule; acquiring one or more images from the illuminating step and analysing the images to determine one or more of the location and orientation of the one or more tension lines; and, perforating at least a portion of the posterior eye capsule with a laser.

In another aspect, the posterior is perforated along one or more lines which are approximately perpendicular to a main axis of the one or more tension lines.

### BRIEF DESCRIPTION OF THE DRAWINGS

FIG. 1 illustrates before and after depictions of a laser capsulotomy.

FIG. 2 illustrates the overall structure of the device for accomplishing the objects of the present invention.

FIG. 3 illustrates an example of the laser firing locations during a laser capsultonomy.

FIG. 4 is a flow chart of events illustrating the operation of the device of the present invention.

### DETAILED DESCRIPTION

With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

Before explaining at least one embodiment of the invention in detail, it is to be understood that the invention is not limited in its application to the details of construction and the arrangement of the components set forth in the following description or illustrated n the drawings. The invention is applicable to other embodiments or of being practiced or carried out in various ways. Also, it is to be understood that the phraseology and terminology employed herein is for the purpose of description and should not be regarded as limiting.

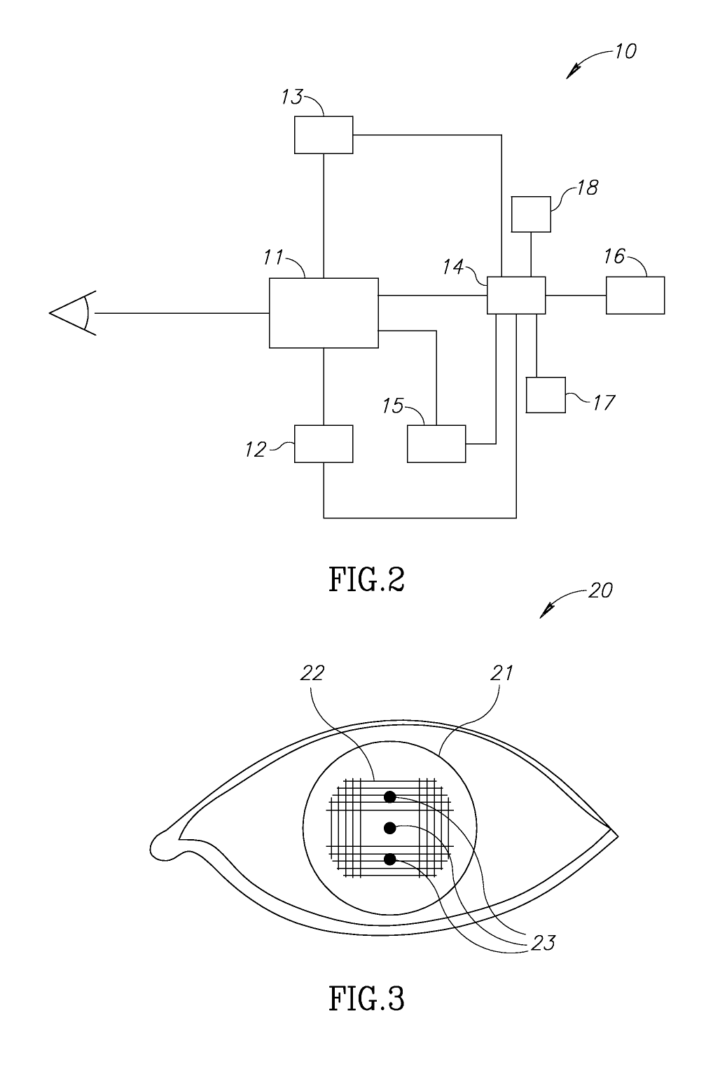

Referring now to FIG. 2, a treatment device embodiment in the present invention is shown at 10. The device comprises a slit lamp 11 and a treatment laser unit 12 optically linked to or integrated with slit lamp 11. Laser 12 is configured to deliver a laser beam into the main optical axis of slit lamp 11. Laser module 12 may be for example an Nd:YAG laser or a diode laser and it may include at least one visible diode laser or lamp source which is configured to use as an aiming beam. Device 10 further comprises digital camera 13 which is optically connected, directly or indirectly, to slit lamp 11 field of view and is configured to image at least a portion of the posterior capsule of a patient's eye. Device 10 may further comprise a controller 14 which is configured to control the operation of laser 12 and camera 13 based on inputs entered by a physician through user interface 16. Optionally user interface 16 may comprise a foot switch and or a touch screen display. Device 10 may further include a dedicated illumination module 15 which is configured to illuminate at least a portion of the posterior capsule of a patient. Illumination module 15 may be integrated into laser module 12 or may further be it laser module 12 itself. Illumination module 15 comprises a light source such as a laser, a lamp, a diode or a LED. Such light sources are configured to illuminate a patient's eye and more particularly at least a portion of a patient's posterior capsule in such a way that it reveals or enhances the contrast between tension lines exist on a posterior capsule of a patient and their surroundings. Illumination module 15 may also light tension lines on the posterior capsule of a patient in such a way that it will reveal or enhance the spatial orientation of such tension lines. Illumination module 15 may light a patient's eye directly or through, for example, a slit lamp 11.

Device 10 may further comprise display module 17. Display module 17 may be for example a screen, a touch screen which is also configured to receive physician inputs or a head-up display. According to one embodiment of the present invention, a head-up display may share the same field of view with slit lamp 11. Device 10 may further include a memory module 18 which may contain previously stored images of a patient's eye taken in different modalities and dates. Controller module 14 may register such images together or with a real time image acquired by camera 18. Images can be uploaded offline into device 10 prior to a laser treatment and stored in memory module 18 in such a way that a physician can view and manipulate such images through user interface 16 prior or during treatment. Such manipulation may include marking planed treatment pattern, location or optical characteristics. Images from different modalities such as for example OCT, fundus camera or confocal laser microscopy may be viewed and registered based on eye anatomy into a single frame of scaled image. Moreover, image registration may further include registering such images with an online image captured by cameral 18. Controller module 14 is configured to allow the physician to switch views from one modality to another or between different combinations of different modalities overlapped and registered into a common scaled frame.

Tension lines on the posterior capsule reflect a mechanical state of the cells, the fibrous layers affecting the opacification of the capsule and the capsule along these lines. Changes in the mechanical states of these organs affect the optical characteristic of such organs and hence can be detected by measuring changes of absorption, reflection, scattering or interference of light targeting such organs as a function of time, light intensities or frequencies. Controller 14 is configured to control illumination module 15 so that illumination intensities or frequencies can be controlled in order to optimize the contrast and visibility of such tension lines. Controller 14 may further include image processing software to further improve the contrast and visualization of such tension lines in an acquired image. Controller 14 may be further configured to synchronize illumination module 15 and camera module 13 in order to capture a series of images of the posterior capsule at different illumination conditions as delivered by illumination module 15. Such images are then stored in memory module 18 and are available for further image processing and displaying. Controller 14 may have a processor (not shown) to process commands from the controller and cause, for example, illumination of the posterior capsule and the firing of the laser. Instructions to perform this and other functions under the control of the controller may be stored in a non-transitory computer-readable medium.

By identifying tension lines by system 10 and providing inputs through user interface module 16, a physician may operate treatment laser module 12 and target areas on the posterior capsule having increased density of tension lines in such a way that it will shoot the laser along lines which are approximately perpendicular to the main linear axis of such tension lines.

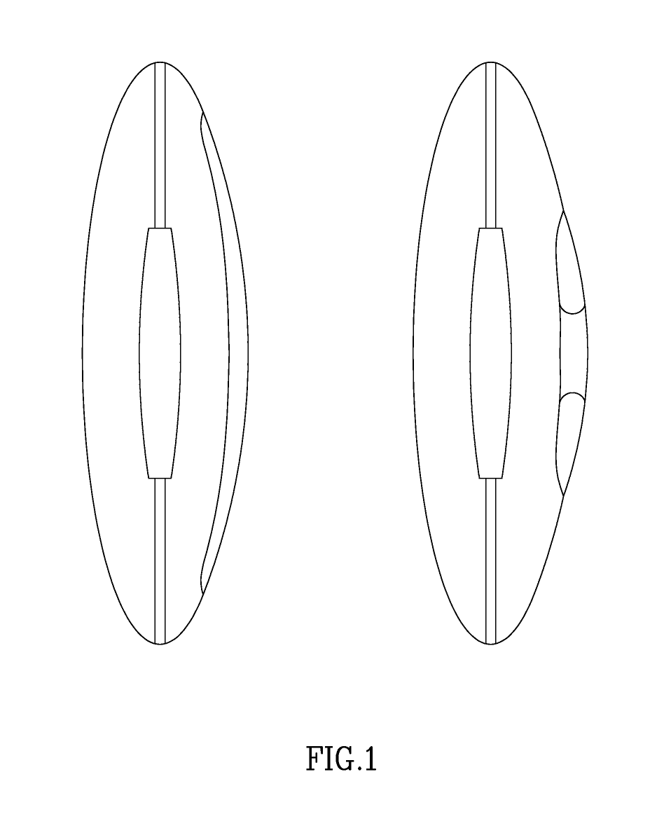

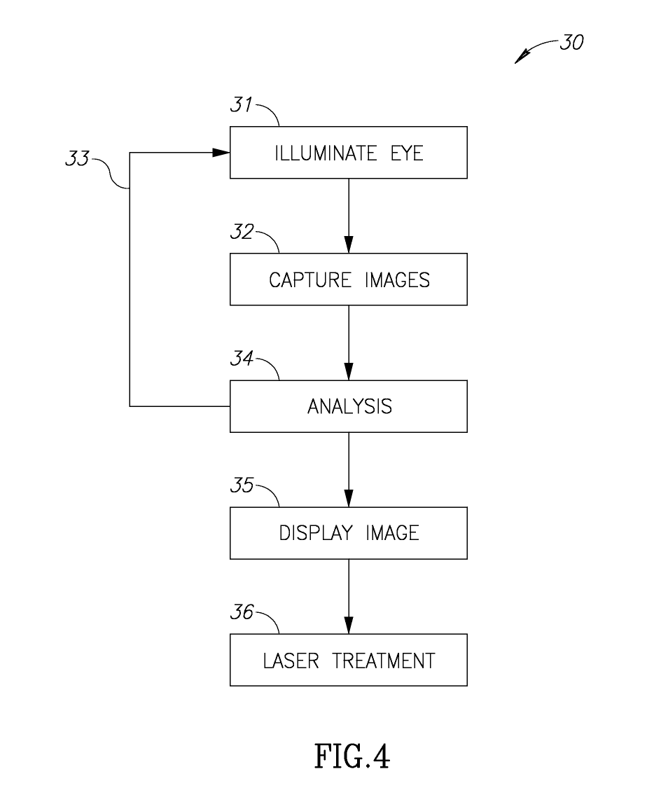

A method of treating a secondary cataract in a patient eye using the laser capsulotomy device of FIG. 1 is shown with reference to FIGS. 3 and 4. FIG. 3 shows a patient's eye 20 having a posterior capsule 21 and tension lines 22. Laser 12 shoots at least one laser treatment pulse 23 across tension lines 22. As shown in FIG. 4, the method includes acquiring an image of at least a portion of a patient's posterior capsule, displaying at least a portion of the tension lines in such image, targeting a treatment laser to posterior capsule of such patient and shooting at least one laser pulse aiming to perforate the posterior capsule. As illustrated at 30, at step 31 at least a portion of the posterior capsule of the patient's eye is illuminated. Illuminating the posterior capsule may be done by changing optical characteristics of the illuminating light such as intensity, duration, frequency, angle or pulsation. At step 32 an image of at least a portion of the posterior capsule of the patient's eye is acquired. As illustrated by arrow 33, multiple images of the posterior capsule can be acquired at different illumination profiles. At analysis step 34, optical changes can be identified based on the analysis of at least one of the absorption, reflection, scattering or diffraction of the illuminated or back scattered light, and the location or orientation of tension line on the posterior capsule can be identified. Image processing software may further enhance the identification or location and orientation of such tension lines. At step 35, a resulting image is displayed to a physician, and at step 36 the treatment laser is used to perforate the posterior capsule of a patient's eye along at least one line which is approximately perpendicular to the main axis of the tension lines.

In the above description, an embodiment is an example or implementation of the invention. The various appearances of “one embodiment”, “an embodiment” or “some embodiments” do not necessarily all refer to the same embodiments.

Although various features of the invention may be described in the context of a single embodiment, the features may also be provided separately or in any suitable combination. Conversely, although the invention may be described herein in the context of separate embodiments for clarity, the invention may also be implemented in a single embodiment.

Furthermore, it is to be understood that the invention can be carried out or practiced in various ways and that the invention can be implemented in embodiments other than the ones outlined in the description above.

Meanings of technical and scientific terms used herein are to be commonly understood as by one of ordinary skill in the art to which the invention belong, unless otherwise defined.

What we claim is:

1. A method of performing a laser capsulotomy on a posterior capsule, comprising:
providing a laser;
illuminating at least a portion of the posterior capsule;
acquiring at least one image of the illuminated posterior capsule;
analyzing the image of the acquired image;
identifying optical changes based on one or more of: absorption, reflection, scattering of illuminated or back-scattered light;
identifying one or more of the location and orientation of one or more tension lines formed on the posterior capsule;
displaying one or more images of the tension lines identified; and,
perforating at least a portion of the posterior capsule along at one or more of the tension lines.

2. The method of claim 1, further comprising the step of acquiring a plurality of images at different illumination profiles during the step of acquiring an image of the illuminated posterior capsule.

3. The method of claim 2, wherein the step of acquiring an image of the illuminated posterior capsule includes changing the optical characteristics of the illuminating light.

4. The method of claim 3, wherein the changing of optical characteristics include one or more of changing: intensity, duration, frequency, angle or pulsation.

5. The method of claim 4, further comprising processing of the one or more optical characteristics to produce multiple images of the posterior capsule based on the different illumination characteristics.

6. The method of claim 1, wherein the step of perforating the posterior capsule comprises perforating along one or more lines which are approximately perpendicular to a main axis of the one or more tension lines.

7. Apparatus for performing a laser capsulotomy on a posterior eye capsule comprising:
a laser source;
a controller to control the laser source;
the controller having a processor and a non-transitory computer-readable medium;
the non-transitory computer-readable medium storing instructions that, when executed by the processor, cause the processor to:
illuminate at least a portion of the posterior capsule;
acquire at least one image of the illuminated posterior capsule;
analyze the image of the acquired image;
identify optical changes based on one or more of: absorption, reflection, scattering of the illuminated or back-scattered light;
identify one or more of the location and orientation of one or more tension lines formed on the posterior capsule;
display one or more images of the tension lines identified on a display; and,
perforate at least a portion of the posterior capsule along at one or more of the tension lines.

8. The apparatus of claim 7 wherein the laser source is a Nd:YAG laser.

9. The apparatus of claim 7, wherein the posterior capsule is perforated along one or more lines which are approximately perpendicular to a main axis of the one or more tension lines.

10. A method for performing a laser capsulotomy on a posterior eye capsule comprising:
illuminating the posterior eye capsule to identify one or more tension lines formed in the posterior eye capsule;
acquiring one or more images from the illuminating step and analysing the images to determine one or more of the location and orientation of the one or more tension lines; and,
perforating at least a portion of the posterior eye capsule with a laser.

11. The method of claim 10, wherein the posterior is perforated along one or more lines which are approximately perpendicular to a main axis of the one or more tension lines.
